(19) United States
(12) Patent Application Publication
(10) Pub. No.: US 2010/0305482 A1
(43) Pub. Date: Dec. 2, 2010

(54) SENSOR APPLICATION COMPRISING A CARRIER STRUCTURE AND A SENSOR STRIP, CARRIER STRUCTURE, SENSOR STRIP AND METHOD FOR OPERATING THE SENSOR APPLICATION

(76) Inventors: Dirk David Goldbeck, Munchen (DE); Tobias Happel, Berlin (DE)

Correspondence Address: King & Spalding LLP, 401 Congress Avenue, Suite 3200, Austin, TX, 78701, US

(21) Appl. No.: 12/741,221

(22) PCT Filed: Nov. 4, 2008
(86) PCT No.: PCT/EP08/64950

§ 371 Date: Jul. 19, 2010

(30) Foreign Application Priority Data
Nov. 8, 2007 (DE) 10 2007 054 305.2

(51) Int. Cl. A61B 5/11; H04Q 5/22

(52) U.S. Cl. 600/595; 340/10.1

(57) ABSTRACT

A sensor application (11) has modules consisting of a carrier strip (13) and a sensor strip (12). The sensor strip (12) is fixed to the carrier strip (13) which, in turn, is fixed, for example, to the back of a human proband. In this way, the spinal curvature can be determined, the carrier strip (13) and the sensor strip (12) being elastically embodied for this purpose. The carrier strip (13) has a data memory (22), for example an RFID label on which relevant information is stored. The data can be evaluated by an evaluation device (14), and the sensor strip (12) is only released when the carrier strip is suitable for the sensor strip used. In this way, defective functions and measuring errors are advantageously avoided.

### CROSS-REFERENCE TO RELATED APPLICATIONS

This application is a U.S. National Stage Application of International Application No. PCT/EP2008/064950 filed Nov. 4, 2008, which designates the United States of America, and claims priority to German Application No. 10 2007 054 305.2 filed Nov. 8, 2007, the contents of which are hereby incorporated by reference in their entirety.

### TECHNICAL FIELD

The invention relates to a sensor application for a moveable body. This sensor application has an elastic carrier structure with a connecting surface, which can be fastened to the surface of a body by way of this connecting surface. The sensor application also has a flexible sensor strip, which can be held by the carrier structure such that the sensor strip follows the contour of the surface. The invention further relates to a flexible sensor strip and/or a carrier structure, with these components being suited to use in the sensor application.

### BACKGROUND

A sensor application and/or a sensor strip and a carrier structure of the type cited in the introduction can be found in U.S. Pat. No. 7,033,281 B2 for instance. According to this a sensor application is used in order to determine the bending angle of the elbow for instance. The moveable body is formed here by the human arm. To enable a movement of the elbow, the carrier structure must be embodied elastically for the sensor strip. According to U.S. Pat. No. 7,033,281 B2, this is ensured by a collar, which is pushed over the elbow joint and covers a part of the upper arm and/or the lower arm. The elasticity of the collar allows a movement of the elbow joint and at the same time ensures that the collar rests closely against the arm of the proband, in other words follows the contour of the surface of the arm. This ensures that a sensor strip, which is placed inside the collar, can then be held by the collar following the contour of the surface as a result of its flexibility.

Naturally, the carrier structure does not have to be embodied as a collar. It is also conceivable to embody the carrier structure as a strip-type adhesive plaster, which can receive the flexible sensor strip. Other active mechanisms such as suction cups, magnetic forces or electrostatic forces are likewise conceivable as a function of the properties of the body to be measured.

### SUMMARY

For an adjustment to different body sizes and/or for hygiene reasons, the sensor application may, when applied to the human body, be constructed from two separate structural units, namely the sensor strip and the carrier structure. According to various embodiments, a sensor application etc. for use in these suitable carrier structures and sensor strips can be specified, the use of which guarantees a reliable and fault-free operation.

According to an embodiment, a sensor application for a moveable body may comprise an elastic carrier structure with a connecting surface, which can be fastened to the surface of the body by the connecting surface and a moveable sensor strip, which is held by the carrier structure following the contour of the surface, wherein the sensor strip and the carrier structure are connected to one another by means of a detachable connection, with the carrier structure having a data storage device, in which a code is stored and which has a transmit interface for the code and with the sensor strip having a switching unit, with which the sensor function can be disabled and enabled and which has a receive interface for an enabling and disabling signal obtained by evaluating the code.

According to another embodiment, a sensor application for a moveable body may comprise an elastic carrier structure with a connecting surface, which can be fastened to the surface of the body by way of the connecting surface and a flexible sensor strip, which is held by the carrier structure following the contour of the surface, wherein the sensor strip and the carrier structure are connected to one another by means of a detachable connection, with the carrier structure having a data storage device, in which a code is stored and which has a transmit interface for the code and with the sensor strip being connected to a control device, which has a receive interface for the code and an evaluation unit for the code for generating an enabling or disabling signal, with which the sensor function can be disabled and enabled.

According to yet another embodiment, an elastic carrier structure with a connecting surface, which can be fastened to the surface of a moveable body by way of the connecting surface may comprise a receptacle for a flexible sensor strip, which can be held by the carrier structure following the contour of the surface, wherein the carrier structure has a detachable connecting structure for fastening the sensor strip and a data storage device, in which a code is stored and which is equipped with a transmit interface for the code.

According to a further embodiment of the elastic carrier structure, the data storage device and the transmit interface can be realized by means of an RFID transponder. According to a further embodiment of the elastic carrier structure, the code may contain information relating to the condition of the sensor strips to be used. According to a further embodiment of the elastic carrier structure, the code may contain a serial number. According to a further embodiment of the elastic carrier structure, the code may contain a use-by date.

According to yet another embodiment, a flexible sensor strip, which can be held by an elastic carrier structure, which can be fastened for its part to a connecting surface on the surface of a moveable body, may comprise a detachable connecting structure relative to the fastening on the carrier strip following the contour of the surface and a switching unit, with which the sensor function can be disabled and enabled and which is equipped with a receive interface for an enabling or disabling signal obtained by evaluating a code stored in the carrier structure.

According to a further embodiment of the flexible sensor strip, a receive facility for the code may be integrated in the sensor strip, said receive facility being connected to an evaluation unit integrated in the sensor strip in order to generate the enabling or disabling signal, with this communicating with the receive interface or the switching unit. According to a further embodiment of the flexible sensor strip, the receive facility may consist of a read device for RFID transponders.

According to yet another embodiment, a method for operating a sensor application for a moveable body, comprises: an elastic carrier structure is fastened to a connecting surface on the surface of the body, a flexible sensor strip is fastened to the carrier structure following the contour of the surface and the measured values describing the contour of the body are generated, wherein the sensor strip and the carrier structure are connected to one another by a detachable connection, with the carrier structure having a data storage device, in which a code is stored, which is read out by way of a transmit interface and evaluated, and with the sensor strip only being enabled for the generation of measured values if the evaluation of the code indicates a suitability of the carrier structure to the sensor strip and is otherwise disabled.

### BRIEF DESCRIPTION OF THE DRAWINGS

Further details of the invention are described below with reference to the drawing. The same or corresponding drawing elements are provided with the same reference characters and are only explained more than once if differences result between the individual figures, in which;

FIG. 1 shows a perspective view of an exemplary embodiment of the sensor application, comprising an exemplary embodiment of the sensor strip and an exemplary embodiment of the carrier structure,

FIG. 2 shows a side view of an alternative embodiment of the exemplary embodiment according to FIG. 1,

FIG. 3 shows an exemplary embodiment of the sensor strip, with the circuit for the evaluation of the code being shown as a block diagram and

FIG. 4 shows a block diagram of another exemplary embodiment of the evaluation of the code in accordance with the method.

### DETAILED DESCRIPTION

According to various embodiments, the sensor strip and the carrier structure are connected to one another by means of a detachable connection, with the carrier structure having a data storage device, in which a code is stored and which comprises a transmit interface for the code and with the sensor strip having a switching unit, with which the sensor function can be disabled and enabled. Furthermore, the sensor strip has a receive interface for an enabling or disabling signal obtained by evaluating the code. To ensure communication between the carrier structure and the sensor strip, an evaluation unit is required, which does not however have to be embodied as part of the sensor strip or the carrier structure. This communicates with the transmit interface of the carrier structure in order to read in the code. By evaluating the code, provided there is a trouble free usage situation of the sensor application, an enabling signal can be transmitted to the receive interface of the sensor strip. If the evaluation of the code indicates however that a trouble-free use of the sensor application is not possible, the sensor strip is disabled and a faulty measurement is prevented in this way. This faulty state can be indicated to a user by way of an output facility, for instance a red LED.

The terms transmit interface and receive interface are to be understood in the broadest sense in conjunction with this invention. Both the transmission and reception can be realized wirelessly, for instance as a radio or infrared connection. Other wired transmission mechanisms (electrical, optical) are however also possible. In the latter case, signals are also namely sent and/or received.

According to an alternative solution of the object, provision is made, for a sensor application as cited in the introduction, for the sensor strip and the carrier structure to be connected to one another by means of a detachable connection, with the carrier structure having a data storage device, in which a code is stored. Furthermore, the data storage device has a transmit interface for the code. The sensor strip is connected to a control device, which has a receive interface for the code and an evaluation unit for the code for generating an enabling or disabling signal, with which the sensor function can be disabled and enabled. In this case accordingly, an external control device is not needed, since the functional element needed to receive and evaluate the code stored in the carrier structure is completely integrated in the sensor strip. This gives the advantage of short transmission paths and, as a result of the integration into a housing, avoidance of fault sources which can occur for instance as a result of inadequate cabling.

When disabling and enabling signals are referred to within the context of this invention, these signals can also be generated in the simplest cases as binary signals by supplying the signal line with a current or by switching off this current. In this simple case, the switching unit is a relay, which is switched on for instance when a current is present in the said signal line (enabling signal) and thus permits a measurement by means of the sensor strip and which is switched off if a current (disabling signal) is not present in the signal line and in this way prevents a measurement with the sensor strip.

The object is also achieved by an elastic carrier structure of the type cited in the introduction, in which a detachable connecting structure is provided in order to fasten the sensor strip and a data storage device. A code is stored in the data storage device, which can be read out by way of a transmit interface.

The object is also achieved by a flexible sensor strip of the type cited in the introduction, with this sensor strip having a detachable connecting structure relative to the fastening following the contour of the surface of the body to be measured on the carrier strip and a switching unit. With the switching unit, the sensor function can be disabled and enabled, with the switching unit being equipped with a receive interface for an enabling or disabling signal to be used to disable and enable the switching unit. The enabling or disabling signal is obtained by evaluating a code stored in the carrier structure.

The code contains user-relevant data, which, in conjunction with the sensor application used, can be evaluated as necessary prerequisites for a reliable operation. An enabling or disabling signal can be generated by evaluating the code in order to prevent a measurement in the case of an impermissible operating state. This advantageously achieves improved reliability against the generation of measuring errors, which, with the use of the sensor application on the human or animal body for diagnostic purposes for instance, ensures the necessary reliability of the measuring results and prevents false diagnoses.

In accordance with a special embodiment, the code can contain information relating to the properties of the sensor strips to be used for instance. This is then particularly advantageous if a modular system comprising different sensor strips and carrier strips is used, with only certain sensor strips and certain carrier strips being allowed to be combined with one another. In this case, information relating to the sensor strip used must also be processed, which is stored in a further data storage device in the sensor strip. Mechanical characteristic values (bending stiffness of the sensor strip) or geometric characteristic values (length and/or width of the sensor strip) can be used as information relating to the condition of the sensor strips to be used. If the sensor application is used for instance to measure the spinal curvature on the back of a human proband, it may be necessary, as a function of the body size, to stock sensor strips and carrier structures of different lengths. By storing information relating to the length of the sensor strips to be used with a specific carrier structure, it is possible to ensure that an excessively short sensor strip is not used in a carrier structure for instance which has to be used per se with a sensor strip of a different length.

According to another embodiment of the carrier structure, the code can also contain a serial number. This advantageously makes it possible to clearly assign the used carrier structures on the basis of the serial number stored therein. Provision can be made for instance for the carrier structure to only be used once, which may for instance represent a requirement in the medical field for hygiene reasons. In this case, the evaluation unit used to evaluate the code requires an additional data storage device, in which the serial numbers of the used carrier structures can be stored for instance. By comparing the serial number of a currently used carrier structure with the serial numbers located in the storage unit, an unauthorized multiple usage is identified and the function of the sensor strip is disabled.

A further embodiment provides for the code to contain a use-by date. In particular in the medical field, the use of carrier structures can be linked to a permissible time frame. Similarly to dressing material for instance, the carrier structures, which are preferably applied to the skin by means of an adhesive layer in the manner of plaster, can lose their effectiveness and/or skin tolerance as a result of ageing. By evaluating the code, the evaluation unit can then determine whether the use-by date of the used carrier structure has been exceeded. To this purpose, the evaluation unit must have access to current data, which can be realized for instance with an internal calendar function. In the event that the use-by data of the carrier function used has been exceeded, the function of the sensor band is disabled.

Note should finally be taken of the use of a code also being advantageous in that unauthorized products, for instance counterfeit products, cannot be used as carrier structures. These are usually produced without any coding, so that the evaluation unit does not receive any response to the code question. In this case, the function of the sensor strip can likewise be disabled. This ensures that only authorized carrier structures are used with the sensor strip. This is conducive to reliably and accurately generated measuring values, since the carrier structure has to be optimally adjusted to the sensor strips used in respect of its mechanical properties, in order to achieve an optimal measuring result. It must be ensured for instance that the sensor strip is also subsequently mounted on the carrier structure following the surface of the body, during a change in shape of the body, since a removal of the sensor strip would result in measuring errors for instance.

It is particularly advantageous if the data storage device and the transmit interface of the carrier structure are realized by means of an RFID transponder. Accordingly, a special embodiment of the sensor strip is produced in that the receive facility consists of a read device for RFID transponders. However, the read facility can also be realized by an external evaluation device. The RFID technology is advantageous in that the outlay for the electronic encoding of the carrier structures, which frequently has to be exchanged, can be kept within a limit. This has a positive effect on the profitability of production. The RFID transponders used in the carrier structure manage without any external power supply if the energy is supplied by means of the read device via the antenna of the RFID transponder. Furthermore, RFID transponders can also be advantageously embodied to be very flat, so that they can be easily integrated in the carrier structure. This maintains a predominantly flat structure, which represents an important requirement for a connection following the surface of the body. Particularly with the application of the carrier structure on the human body, the wearing comfort is herewith also advantageously improved. Finally, the wireless transmission of the code likewise results in an improvement in the wearing comfort.

According to various embodiments of a method for operating a sensor application for a moveable body, the following steps are executed. An elastic carrier structure with a connecting surface is fastened to the surface of the body. A flexible sensor strip is subsequently fastened to the carrier structure following the contour of the surface. The measured values describing the contour of the body are generated.

A method of this type is described in U.S. Pat. No. 7,033,281 B1 as cited in the introduction. This results in the object of specifying a method for operating the sensor application as cited in the introduction, in which comparatively reliable measured values can be generated with comparatively fewer measuring errors.

According to embodiments of a method, the sensor strip and the carrier structure are connected to one another by means of a detachable connection, with the carrier structure having a data storage device in which a code is stored, which is read out and evaluated by way of a transmit interface and with the sensor strip only being enabled for the generation of measured values if the evaluation of the code indicates a suitability of the carrier structure to the sensor strip and otherwise being disabled.

The advantages already mentioned are achieved using the method according to various embodiments. The improvement in the measuring quality, in other words a reduction in measuring errors and/or a higher reliability in the generation of the measured values is achieved in that, in instances in which a faulty operation of the sensor application has to be expected as a result of an evaluation of the code, the function of the sensor strip is not enabled but is instead disabled. In these instances, measuring values are not generated. Instead, the error can be output by way of a suitable output facility. This output facility can consist of an LED for instance, a display is also possible, which can output additional information relating to the type of error.

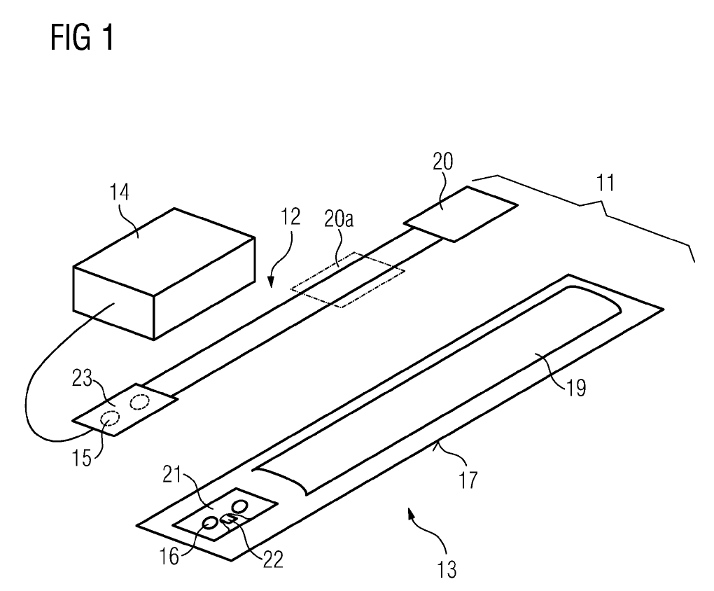

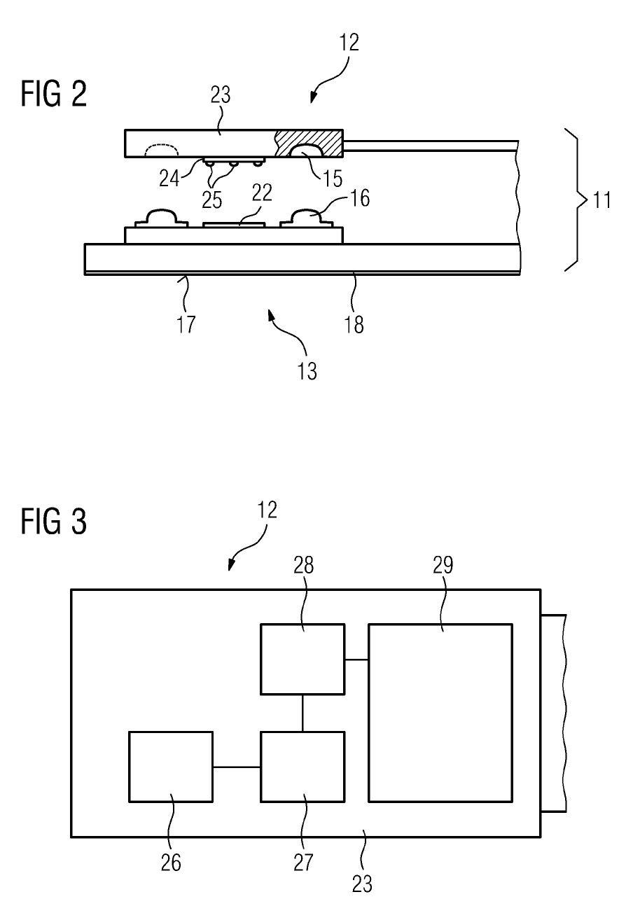

According to FIG. 1, a sensor application 11 is shown, which consists of a sensor strip 12 and a carrier structure 13. An evaluation unit 14 is also provided, to which the sensor application 11 is connected (contacting of the sensor strip 12). The sensor strip 12 can be connected in a form-fit fashion to a corresponding connecting structure 16 (press studs) of the carrier strip by means of a connecting structure 15 (indentations), as a result of which the sensor application is combined to form a structural unit. The sensor application can be fastened to a body (not shown) to be measured by way of a connecting surface 17, said body consisting of the back of a human proband for instance. An adhesive layer 18 (see FIG. 2) can be used here for instance. To ensure that after fastening the sensor application 11 to the body, the flexible sensor strip 12 follows the contour of the surface of the body and does not protrude from the carrier structure 13, an elastic pocket 19 is also formed on the carrier structure 13, into which the free end 20 of the sensor strip is inserted before the connecting structures 15, 16 are connected to one another. The carrier structure 13 itself consists of a flexible strip, which, in addition to a flexibility, also has a length extension, with a lateral contraction being prevented by a suitable tissue structure when lengthening the strip. The sensor strip can slide unimpeded into the pocket if a relative movement is needed between the sensor strip 12 and the carrier structure 13 as a result of a bend impressed into the sensor application 11.

The connecting structure 16 is attached to a rigid base plate 21, in order also to ensure a reliable connection between the carrier strip 13 and the sensor strip 12 if the carrier structure 13 is elastically deformed. The base plate 21 is used at the same time to receive a data storage device 22 in the form of a RFID transponder, which can be accommodated in a protected fashion between the base plate 21 and the carrier structure 13. This is activated by the evaluation unit 14, so that the information stored in the data storage device 22 can be read out wirelessly. As a function of this data, the evaluation unit will generate an enabling or disabling signal and forward it to the sensor strip 14. The function of the sensor strip is only activated with the presence of an enabling signal. If an excessively short sensor strip is used for instance (indicated by the end 20a of the sensor strip 12), a comparison of the data read out from the data storage device 22 with the data which is stored in a further data storage device (not shown) is stored in the sensor strip and made available by way of the connection of the evaluation unit 14, results in the evaluation unit 14 identifying the use of an incorrect sensor strip and generating a disabling signal, which suspends the function of the excessively short sensor strip 12.

FIG. 2 shows an exemplary embodiment, in which the sensor strip 12 in the end housing 23 (also identifiable in FIG. 1) has an evaluation unit (not shown in further detail). It is therefore possible to dispense with the external evaluation unit 14 according to FIG. 1. A contact plate on the base plate 21 can be used as a data storage device 22, on which a certain contact pattern is realized in an array of 3×3 contacts. This contact pattern corresponds to a data record, which can be detected by a corresponding contact plate 24 at the end housing 23 by way of spring contacts. The spring contacts 25 are connected (not shown) to the evaluation unit (likewise not shown) so that this can further process the determined data.

The end housing 23 of the sensor strip 12 according to FIG. 3 is represented with a block diagram of the used elements of a circuit for evaluating the data stored in the carrier strip. A receive facility 26 is initially provided, which activates a data storage device (not shown) in the form of an RFID transponder and reads in the code by way of a receive interface (not shown in further detail) (embodied as a radio interface). This is forwarded to an evaluation facility 27, in which the information contained in the code is decoded and evaluated. As a function of the result of the evaluation, the carrier strip is either classified as suitable or unsuitable. If the carrier strip is suitable, an enabling signal is sent to a switching unit 28, so that the switching unit 28 releases a driver electronics system 29 for the sensor strip 12 and measuring values can thus be generated. On the other hand, the evaluation facility 27 transmits a disabling signal to the switching facility 28, with the switching facility then deactivating the driver electronics system 29.

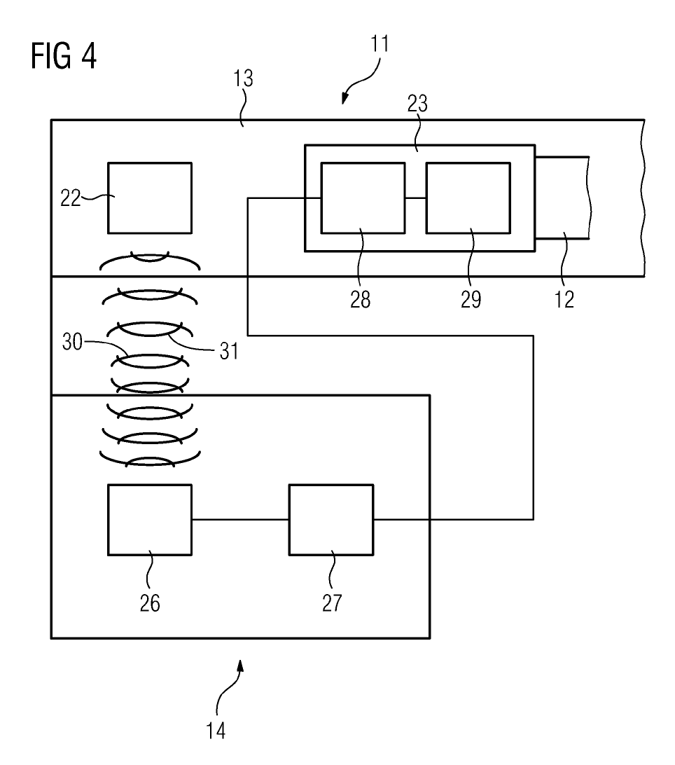

The method can also be reproduced in accordance with FIG. 4, with an external evaluation facility 14 according to FIG. 1 being used here. In the evaluation facility 14, the actual evaluation facility 27 is realized by means of a module. In this variant, only the switching unit 28 is positioned in the end housing 23 of the sensor strip 12. This is connected to the evaluation facility 27. Otherwise, the configuration corresponds to that according to FIG. 3 and functions similarly. FIG. 4 also shows the data storage device 22 in the form of the RFID transponder. An activation signal 30 is also indicated, which is transmitted by the receive facility 26 and activates the data storage device 22 and a code signal 31, which transmits the data storage device 22 and which contains the information stored in the code.

What is claimed is:

1. A sensor application for a moveable body, comprising
an elastic carrier structure with a connecting surface, which can be fastened to the surface of the body by the connecting surface and
a moveable sensor strip, which is held by the carrier structure following the contour of the surface,
wherein
the sensor strip and the carrier structure are connected to one another by means of a detachable connection, with
the carrier structure having a data storage device, in which a code is stored and which has a transmit interface for the code, and
the sensor strip having a switching unit, with which the sensor function can be disabled and enabled and which has a receive interface for an enabling and disabling signal obtained by evaluating the code.

2. A sensor application for a moveable body, comprising
an elastic carrier structure with a connecting surface, which can be fastened to the surface of the body by way of the connecting surface, and
a flexible sensor strip, which is held by the carrier structure following the contour of the surface,
wherein
the sensor strip and the carrier structure are connected to one another by means of a detachable connection, with
the carrier structure having a data storage device, in which a code is stored and which has a transmit interface for the code, and
the sensor strip being connected to a control device, which has a receive interface for the code and an evaluation unit for the code for generating an enabling or disabling signal, with which the sensor function can be disabled and enabled.

3. An elastic carrier structure with a connecting surface, which can be fastened to the surface of a moveable body by way of the connecting surface, comprising a receptacle for a flexible sensor strip, which can be held by the carrier structure following the contour of the surface,
wherein
the carrier structure
has a detachable connecting structure for fastening the sensor strip, and
a data storage device, in which a code is stored and which is equipped with a transmit interface for the code.

4. The carrier structure according to claim 3, wherein
the data storage device and the transmit interface are realized by means of an RFID transponder.

5. The carrier structure according to claim 3, wherein
the code contains information relating to the condition of the sensor strips to be used.

6. The carrier structure according to claim 3, wherein
the code contains a serial number.

7. The carrier structure according to claim 3, wherein the code contains a use-by date.

8. A flexible sensor strip, which can be held by an elastic carrier structure, which can be fastened for its part to a connecting surface on the surface of a moveable body,
wherein
the sensor strip comprises
has a detachable connecting structure relative to the fastening on the carrier strip following the contour of the surface, and
has a switching unit (28), with which the sensor function can be disabled and enabled and which is equipped with a receive interface for an enabling or disabling signal obtained by evaluating a code stored in the carrier structure.

9. The sensor strip according to claim 8, wherein
a receive facility for the code is integrated in the sensor strip, said receive facility being connected to an evaluation unit integrated in the sensor strip in order to generate the enabling or disabling signal, with this communicating with the receive interface or the switching unit.

10. The sensor strip according to claim 9, wherein
the receive facility consists of a read device for RFID transponders.

11. A method for operating a sensor application for a moveable body comprising the steps of:
fastening an elastic carrier structure to a connecting surface on the surface of the body,
fastening a flexible sensor strip to the carrier structure following the contour of the surface, and
generating the measured values describing the contour of the body,
wherein
the sensor strip and the carrier structure are connected to one another by a detachable connection, with
the carrier structure having a data storage device, in which a code is stored, which is read out by way of a transmit interface and evaluated, and
the sensor strip only being enabled for the generation of measured values if the evaluation of the code indicates a suitability of the carrier structure to the sensor strip and is otherwise disabled.

12. The method according to claim 11, wherein the data storage device and the transmit interface are realized by means of an RFID transponder.

13. The method according to claim 11, wherein the code contains information relating to the condition of the sensor strips to be used.

14. The method according to claim 11, wherein the code contains a serial number.

15. The method according to claim 11, wherein the code contains a use-by date.
